(19) United States
(12) Patent Application Publication
(10) Pub. No.: US 2007/0049854 A1
(43) Pub. Date: Mar. 1, 2007

(54) Weighted back brace

(76) Inventor: Bahman Teimourian, Bethesda, MD (US)

Correspondence Address: PATTON BOGGS LLP, 8484 WESTPARK DRIVE, SUITE 900, MCLEAN, VA, 22102, US

(21) Appl. No.: 11/211,463

(22) Filed: Aug. 26, 2005

(51) Int. Cl. A61F 5/00

(52) U.S. Cl. 602/19

(57) ABSTRACT

A weighted brace is provided with an element for attachment to the body, a weight carrier having at least one pocket, and at least one weight contained in the at least one pocket, the at lease one weight is rigid or semi-rigid, wherein the at least one weight provides support to at least a lower area of the back when worn in close proximity thereto.

### FIELD OF THE INVENTION

The presented invention is directed to wearable weighted braces or supports.

### BACKGROUND OF THE INVENTION

In the following discussion certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Back pain can be a debilitating condition that is suffered by many people. A variety of different devices and techniques have been proposed to provide therapeutic treatment of back pain.

U.S. Pat. No. 5,713,840 discloses a wearable weighted lumbar support. The device includes weights suspended from the wearer's shoulders. The weights are in the form of deformable and pliable rolls that are transversely oriented across the wearer's back.

U.S. Pat. No. 5,067,484 discloses a posture training support with weighted pockets. Multiple straps worn around the wearer's shoulders that position the weighted pockets in the upper back area. The weights are disclosed as being soft and pliable, such as by filling a fabric pocket with pellets.

U.S. Pat. No. 3,888,245 discloses a weighted surgical belt having a posterior portion positioned to confront the sacral and coccygeal vertebrae. The belt must be provided with a relatively complicated shape that fulcrums over the hip bones and positions the weight-carrying posterior portion in the desired location. The weight member is disclosed in the form of a generally square pad having a side that conforms to the shape of the back and a rounded dome-like opposing side.

The previously proposed solutions possess certain drawbacks. Those devices that have soft and pliable weights in order to maximize comfort sacrifice optimal supportive pressure on the spine. Certain devices require complicated systems of straps or complex geometrical configurations to carry the weight. Other devices do not locate the weight in an area of the body that is both unobtrusive to the wearer and yet offers adequate support. Still other devices lack flexibility so that a range of persons having different physical make-ups and physical needs may utilize the device.

### SUMMARY OF THE INVENTION

It is, therefore, an object of this invention to provide a back brace with one or more of the following features and advantages: good support, adequate downward force to promote the desired spinal alignment, a compact construction that is unobtrusive and may be worn under clothing, a simple design that facilitates manufacture and can be readily manipulated or customized to suit the needs of various wearers.

According to one aspect of the present invention, there is provided a weighted brace comprising body attachment means, a weight carrier comprising at least one pocket, and at least one weight at least partially contained within the at least one pocket, the at least one weight is rigid or semi-rigid, wherein the at least one weight provides support to at least a lower area of the back when worn in close proximity thereto.

According to a further aspect, the present invention provides a weighted brace comprising body attachment means, a weight carrier comprising at least one pocket, and at least one weight at least partially contained within the at least one pocket, the at least one weight is rigid or semi-rigid and generally cylindrical, the at least one weight has a height, wherein the at least one pocket of the weight carrier is constructed and disposed to carry the at least one weight such that its elongated longitudinal dimension is substantially vertically oriented, and the elongated longitudinal dimension of the at least one weight substantially conforms to the distance between the T12 and S1 vertebrae of the wearer.

### BRIEF DESCRIPTION OF THE DRAWINGS

The preferred embodiments are illustrated in the drawings in which like reference numerals refer to the like elements and in which:

FIG. 1 is a plan view of a weighted brace formed according to the principles of the present invention

FIGS. 2A-2C are perspective views of alternative constructions of weight which may be utilized in the apparatus of FIG. 1.

FIG. 3 is a plan view of a device constructed according to a first alternative of the present invention.

FIG. 4. is a plan view of a device constructed according to a second alternative.

FIG. 5A is a plan view of a device constructed according to a third alternative embodiment of the present invention.

FIG. 5B is a side view of a device depicted in FIG. 5A.

FIG. 6 is a plan partial cut away view of the placement of a device constructed according to the principles of the present invention and relative to the wearer's body.

### DETAILED DESCRIPTION OF THE INVENTION

As used herein, the term “rigid” is intended to encompass a structure which is substantially inflexible when subjected to stresses and forces normally encountered during use. The term “semi-rigid” is intended to encompass a structure which may be slightly more flexible than a rigid structure, but which does not readily conform to the contour of the back when subjected to stresses and forces encountered during normal use.

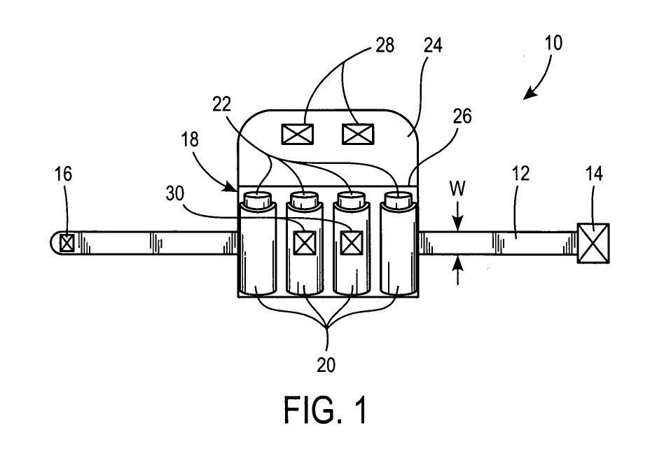

One embodiment of a weighted brace 10 constructed according to an illustrative embodiment, is depicted in FIG. 1. The weighted brace 10 includes body attachment means 12. Body attachment means includes means for suspending weighted brace at the desired location relative to the body of the wearer, in a non-intrusive fashion. Preferably, the body attachments means is a relatively simply shaped and configured structure. For example, the body attachment means of the illustrated embodiment is in the form of a belt. The belt can be of a simple single strap construction having a simple band-like geometry with a constant width W between the first and second ends thereof. The body attachment means can be formed of any suitable material, such as leather, woven natural fibers, woven synthetic fibers, or combination of woven, natural and synthetic fibers. According to the illustrated embodiment, the body attachment means 12 facilitates placement of the weighted brace 10 at the desired location relative to the back of the wearer (as discussed in further detail herein), and due to its simple design, is readily manufactured, and can be worn unobtrusively under the clothing of the wearer. Alternative constructions and modifications to the illustrated body attachment means are contemplated. For example, the body attachment means may comprise multiple straps. For instance, the body attachment means may comprise a belt with suspenders for traveling over the shoulder area of the wearer. As noted above, the belt is preferably constructed of a relatively flexible material. Thus, according to the principles of the present invention, the body attachment means is formed of a material which is relatively flexible when compared to other components of the weighted brace. Thus, the belt does not form the principle mechanism by which the back is supported. Rather, other components of the weighted brace, to be discussed in greater detail below, provide this function. The body attachment means 12 may be provided with securing means for attachment to the wearer. For instance, the securing means may comprise a first means 14 disposed at a first end of the body attachment means, in combination with a second securing means 16 providing at an opposing end of the body attachment means. The securing means can be in the form of any suitable element which releasably retains the weighted brace 10 to the body of the wearer. Suitable elements include a buckle, frictional clasp, snap, hook and loop fastener, or any other similar element common to the art.

One of the main components of the weighted brace 10 is the weight carrier 18. The weight carrier 18 can be formed of any suitable material, such as leather, woven natural fibers, woven synthetic fibers, or a combination of woven natural and synthetic fibers. The weight carrier 18 is formed with at least one pocket 20 which receives at least one weight 22. The at least one pocket 20 can be integrated with the material of the carrier 18, or can be separately fashioned and attached thereto. When separately formed, the pockets 20 may be attached to the weight carrier 18 by any suitable fashion, such as adhesives, stitching, hook and loop fasteners, etc. According to the illustrated embodiment, the weight carrier 18 is provided is provided with a plurality of such pockets 20. Specifically, the illustrated embodiment includes 4 such pockets. However, it should be emphasized, that a number of configurations having different numbers of pockets are well within the confines of the scope of the present invention. The weight carrier 18 may be provided with an optional cover flap 24 which, when folded over along line 26, provides enhanced containment of the weights 22 disposed within the weight carrier 18. Such a construction may be useful when the wearer is expected to undertake rigorous activity or exercise. The cover flap 24 may be secured to the weight carrier 18 by any suitable mechanism. For example, in the illustrated embodiment, the cover flap 24 is provided with closure means 28 and 30 adapted to maintain the cover flap 24 in a closed position. The closure means 28 and 30 can comprise any suitable construction for releasably retaining the closure flap in a desired position. For example, suitable closure means include buttons, clasps, snaps, hook and loop fasteners, etc.

Providing weight carrier 18 with a plurality of pockets designed to accommodate individual weights 22 provides certain advantages. For instance, such a construction provides flexibility when designing a device to be employed by different users having different physical capabilities and physical needs. As can be observed, the cumulative total amount of weight contained in the weighted brace can be varied by removing and/or adding individual weights 22 to the pockets 20. This provides an opportunity to optimize the amount of weight carried by the wearer. While it is expected that the amount of weight to be utilized best determined with the assistance of a physician, a wide range of weight values is possible. For purposes of illustration only, the weighted brace may house a cumulative total weight of 2-12 pounds.

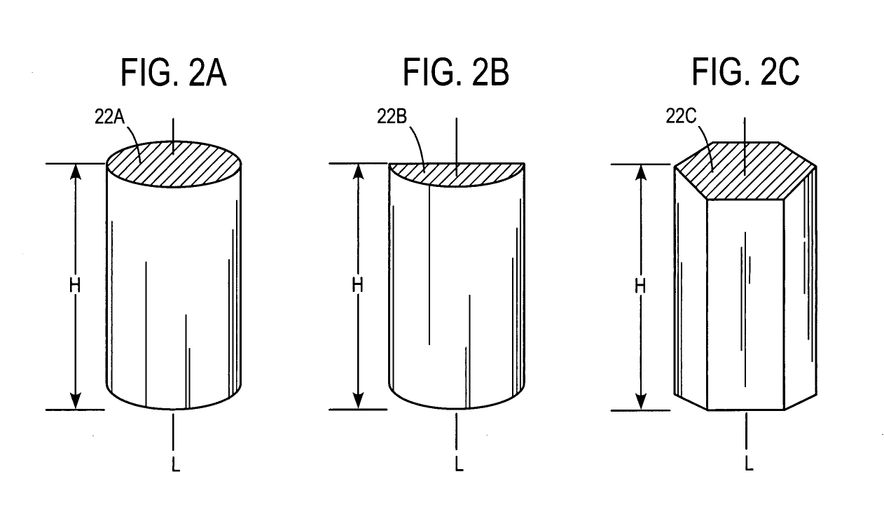

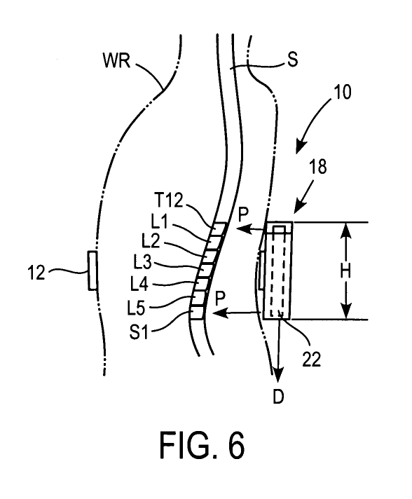

As illustrated in FIGS. 2A-2C, the weights 22 depicted in the illustrative embodiment have a generally cylindrical configuration within an elongated longitudinal dimension, or height H, as measured from the top to the bottom of the weight along the longitudinal axis L thereof. As illustrated in FIG. 1 (see also, FIG. 6) the weight carrier 18 is designed such that the pockets 20 carry the weights 22 in a position such that the elongated longitudinal dimensions of the weights 22 are substantially vertical, or parallel to the spinal column of the wearer, when attached to the body of the wearer during normal and intended use of the weighted brace 10. The weights 22 may be formed of a rigid or semi-rigid material, such that they do not readily conform to the contours of the back when subjected to the normal forces imparted to them during normal use. According to this aspect of the present invention, it has been found by utilizing rigid or semi-rigid weight members, improved supportive pressure may be applied to the desired area of the back of the wearer, thereby improving results in the form of better relief of lower back pain and stresses. It is contemplated by the present invention that, instead of the plurality of separate weights disposed in a plurality of separate pockets, is depicted in FIG. 1, a single unitary weight member may be provided in a single pocket of the weight carrier 18 (not shown). This single weight member can be formed by a plurality of integrated sections. These sections may be frangible, or otherwise separable from the remaining sections such that the amount of weight may be readily varied by simply removing selected sections of the weight member.

It is also noted that the weights may be formed in a number of different suitable shapes or geometries. Samples of such shapes or geometries are illustrated in FIGS. 2A-2C. For example, the generally cylindrical weight 22 can have a substantially round cross section 22A as depicted in FIG. 2A, or a semi circular cross section 22B as depicted in FIG. 2B, or, alternatively, a polygonal cross section 22C as depicted in FIG. 2C. The constructions depicted in FIGS. 2B and 2C provide a flat surface that opposes the back of the wearer, thereby distributing forces over a greater surface area and thereby altering the pressure applied to the back of the wearer. Such configurations may be beneficial for some wearers.

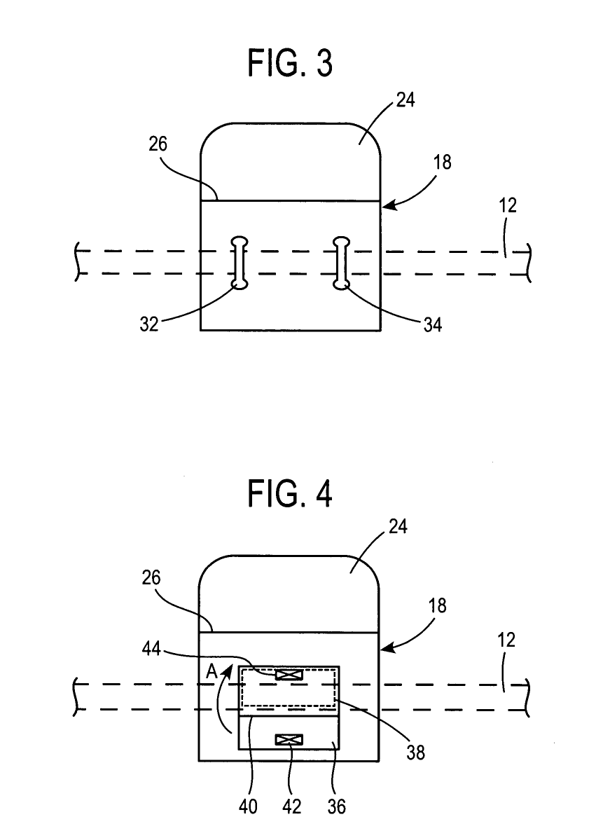

As illustrated in FIGS. 3-5B, the weight carrier 18 is provided with means for securing the weight carrier 18 to the body attachment means 12. Arrangements for securing to weight carrier to the body attachment means can comprise any suitable construction that releasably attaches weight carrier 18 to the body attachment means 12 in a secure manner. Three suitable alternatives are depicted in the above-mentioned figures.

The first alternative is depicted in FIG. 3. According to the illustrative embodiment, a plurality of slots or passages 32 and 34 are provided in the back of a weight carrier 18. The slots or passages 32 and 34 permit threading of the flexible body attachment means 12 therethrough in a slidable manner.

The embodiment illustrated in FIG. 4 depicts a detachable loop construction 36. The detachable loop 36 is attached to the weight carrier 18 by attachment means 38. Attachment means 38 can comprise any suitable construction, such as an adhesive, stitching, hook and loop fasteners, snaps, clasps, etc. Part of the detachable loop 36 is not secured to the back of the weight carrier 18 such that it can be folded up as indicated by arrow A along fold line 40 and secured to the portion of the detachable loop 36 which is attached to the back of the weight carrier 18. Suitable releasable closures means 42 and 44 are provided for this purpose. Releasable closure means can comprise any suitable construction such as hook and loop fasteners, snaps, buttons, clasps, etc. As evident from the embodiment illustrated in FIG. 4, the belt is sandwiched between the portion of the detachable loop 36 which is secured to the back of the weight carrier 18, and the loose flap portion which is folded along line 40 and overlies the belt 12 when secured in a releasable manner via the releasable closure means 42 and 44.

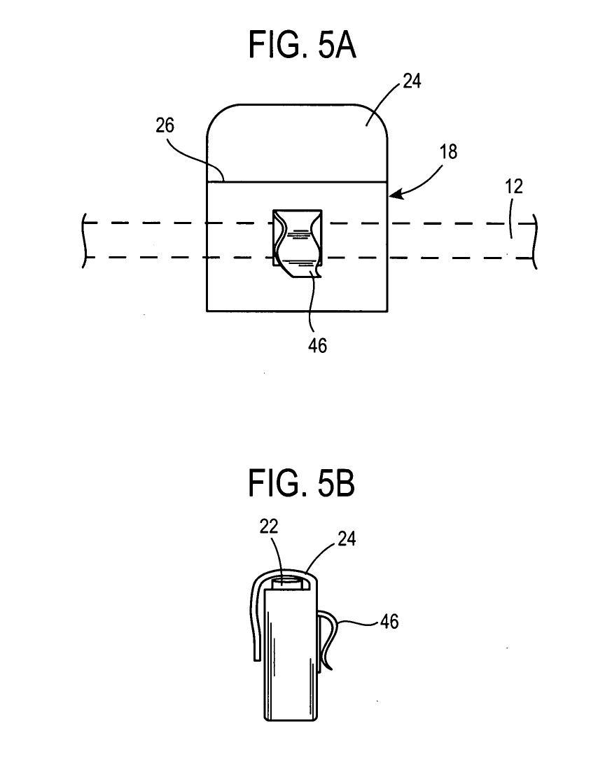

According to the embodiment illustrated in FIGS. 5A-5B, the means for securing the weight carrier 18 to the body attachment means 12 is in the form of a flexible clip 46 which is attached to the back of the weight carrier 18. As is well known in the art, the unattached or free portion of the flexible clip 46 is forced over the body attachment means 12 thereby securing the weight carrier 18 thereto in a releasable manner.

FIG. 6 is a schematic illustration of the weighted brace 10 secured to the back of a wearer WR. As illustrated, the weighted brace 10 is constructed such that when secured to the body of the wearer WR, the elongated longitudinal dimension H of the weights 22 is substantially aligned with, and vertical or parallel to the spinal column S of the back of the wearer. Moreover, the weights 22 are in a position which substantially corresponds to the location of the T12-S1 vertebrae of the wearer WR. Since the weights 22 have a rigid or semi rigid construction, pressure P is advantageously applied in the above-mentioned region of the spinal column S. In addition, the weights 22 generate a generally downward force D, which promotes a beneficial posture in the back of the wearer. As illustrated, the weights 22 may be provided with an elongated longitudinal dimension H which substantially corresponds to the distance between the T12 and S1 vertebrae of the user. Since the distance between the above-mentioned vertebrae can vary from wearer to wearer, it is advantageous that the weighted brace 10 is readily adaptable to meet the needs of different wearers. For example, a physician or other qualified medical personnel can measure the distance between the T12 and S1 vertebrae of the wearer. This measurement is then sent to the manufacturer who modifies the transverse longitudinal dimension H of the weights 22 to specifically fit the back of the wearer of the device. Alternatively, the wearer may purchase standard size weight 22 having a transverse longitudinal dimension H which is greater than the expected distance between the above-mentioned vertebrae, and then shorten this elongated longitudinal dimension to match the measured distance by cutting or other suitable material removal technique. Along these lines, a physician or other qualified medical personnel may also estimate the amount of weight needed by the particular wearer of the device. The manufacturer can then construct a weight, or arrangement of weights, which matches the value provided by the doctor or other qualified medical personnel. Alternatively, the user may purchase a standard sized weight or set of weights, and then select an appropriate weight configuration to match the measurement made by the doctor or other suitable medical personnel.

According to a further alternative, the manufacturer may produce the weighted back brace 10 which comes with weights 22 having an elongated longitudinal dimension H which roughly corresponds to the average distance between the T12 and S1 vertebrae for various categories of users. For example, the weights may be produced in adult large, medium and small standard configurations. Similarly, the amount of weight may also be provided according to similar standards. Thus, the manufacturer may produce a weighted brace in a small configuration with less weight, and medium and large configurations with progressively more weight with longer dimensions H and shorter weights, which generally corresponds to the various categories of wearer types.

While this invention is satisfied by embodiments in many different forms, as described in detail in connection with preferred embodiments of the invention, it is understood that the present disclosure is to be considered as exemplary of the principles of the invention and is not intended to limit the invention to the specific embodiments illustrated and described herein. Numerous variations may be made by persons skilled in the art without departure from the spirit of the invention. The scope of the invention will be measured by the appended claims and their equivalents. The abstract and the title are not to be construed as limiting the scope of the present invention, as their purpose is to enable the appropriate authorities, as well as the general public, to quickly determine the general nature of the invention. In the claims that follow, unless the term “means” is used, none of the features or elements recited therein should be construed as means-plus-function limitations pursuant to 35 U.S.C. §112, ¶6

I claim:

1. A weighted brace comprising:
body attachment means;
a weight carrier comprising at least one pocket; and
at least one weight at least partially contained within the at least one pocket, the at least one weight is rigid or semi-rigid;
wherein the at least one weight provides support to at least a lower area of the back when worn in close proximity thereto.

2. The brace of claim 1, wherein the body attachment means is less rigid than the at least one weight.

3. The brace of claim 1, wherein the body attachment means comprises a belt, the belt comprising a first end and a second end, the belt having a constant width between the first and second ends.

4. The brace of claim 1, wherein the weight carrier comprises a plurality of separate pockets.

5. The brace of claim 1, wherein the weight carrier comprises a cover flap.

6. The brace of claim 1, wherein the weight carrier is constructed from leather, woven natural fibers, woven synthetic fibers, or a combination of woven natural and synthetic fibers.

7. The brace of claim 1, wherein the at least one weight is constructed from a dense metal.

8. The brace of claim 7, wherein the at least one weight is generally cylindrical.

9. The brace of claim 8, wherein the cross section of the cylinder is round, semicircular, or polygonal.

10. The brace of claim 8, wherein the at least one weight has a cumulative total weight of 2 to 12 lbs.

11. The brace of claim 1, wherein the at least one weight has an elongated longitudinal dimension which substantially conforms to the distance between the T12 and S1 vertebrae of the wearer.

12. The brace of claim 8, wherein the at least one pocket of the weight carrier is constructed and disposed to carry the at least one weight such that its elongated longitudinal dimension is substantially vertically oriented.

13. The brace of claim 1, wherein the weight carrier comprises means for securing the weight carrier to the body attachment means.

14. The brace of claim 13, wherein the means for securing comprises at least one of passages, a detachable loop or a clip.

15. A weighted brace comprising:
body attachment means;
a weight carrier comprising at least one pocket; and
at least one weight at least partially contained within the at least one pocket, the at least one weight is rigid or semi-rigid and generally cylindrical, the at least one weight has an elongated longitudinal dimension;
wherein the at least one pocket of the weight carrier is constructed and disposed to carry the at least one weight such that its elongated longitudinal dimension is substantially vertically oriented, and the elongated longitudinal dimension of the at least one weight substantially conforms to the distance between the T12 and S1 vertebrae of the wearer.

16. The brace of claim 15, wherein the body attachment means comprises a belt, the belt comprising a first end and a second end, the belt having a constant width between the first and second ends.

17. The brace of claim 15, wherein the cross section of the cylinder is round, semicircular, or polygonal.

18. The brace of claim 15, wherein the weight carrier comprises means for securing the weight carrier to the body attachment means.

19. The brace of claim 15, wherein the means for securing comprises at least one of passages, a detachable loop or a clip.
